(19) United States
(12) Patent Application Publication
(10) Pub. No.: US 2004/0054266 A1
(43) Pub. Date: Mar. 18, 2004

(54) Computer-implementable vocabulary-based test for a class of language disorder

(76) Inventor: Michacl A. Covington, Athens, GA (US)

Correspondence Address: GLAXOSMITHKLINE, Corporate Intellectual Property - UW2220, P.O. Box 1539, King of Prussia, PA, 19406-0939, US

(21) Appl. No.: 10/660,443

(22) Filed: Sep. 11, 2003

Related U.S. Application Data
(60) Provisional application No. 60/410,237, filed on Sep. 12, 2002.

(51) Int. Cl. A61B 5/00

(52) U.S. Cl. 600/300

(57) ABSTRACT

Method for detecting and objectively measuring a type of language disorder that can occur as a result of mental illness, developmental disorder, brain injury, drug intoxication, or other conditions, but is particularly associated with thought-disordered schizophrenia, and also useful in assessing normal child language development. A subject is given a picture or other stimulus and asked to describe what he or she observes. The description is transcribed and the vocabulary is reviewed manually or by computer to determine completeness of the subject's description of the picture or stimulus.

### CROSS REFERENCE TO RELATED APPLICATIONS

This application claims benefit to U.S. provisional 60/410,237 file Sep. 12, 2002.

### AREA OF THE INVENTION

This invention relates to a method for detecting and objectively measuring a class of language disorder. This kind of language disorder can occur as a result of mental illness, developmental disorder, brain injury, drug intoxication, or other conditions, but is particularly associated with thought-disordered schizophrenia. The method is also applicable to assessing language development in children.

### BACKGROUND OF THE INVENTION

The technique has wide applicability. For expository purposes schizophrenia will focus on herein below. It is a psychosis that can be detected and assessed by this technique.

The psychoses form a group of psychiatric disorders characterized by impaired capacity to recognize external reality: impaired reality testing. While some of the psychoses are of mainly organic origin, many others are less obviously so. Study of the latter group of disorders often, therefore, has to be made in relation to functional aspects such as the type of thought processes used, as evident by the patient's speech.

Several attempts have been made to identify quantifiable factors which may serve as the basis for more objective tests for the diagnosis of psychoses. A number of such studies have been directed towards the use of speech analysis for the diagnosis of schizophrenia and other psychoses. These have invariably focused on the analysis of words as vocal signals [Stassen et al. Psychopathology 24: 88-105 (1991)]. However, the results of such analyses are strongly related to content (i.e. the spoken message) and may be language and culture dependent.

Others have found that the inter-word time intervals contain critical information that makes it possible to carry out diagnostic measurements of psychoses [D Todder, S Shreiber-Avissar, PCT application WO 00/41625, July 2000]. These are unrelated to speech content. Such analyses claim to provide a much purer measurements of the form of speech than were previously available. In addition, such analyses are said to provide a method for determining cognitive development stage in healthy children.

Abnormalities of language are a key aspect of the cognitive disturbances experienced by patients with schizophrenia and may be one of the major barriers to their re-integration into society. This invention provides a new method for detecting and objectively measureing certain language disorders. It is accomplished by giving the subject something to describe, such as a picture, then scoring the description as to whether it mentions all of the salient attributes, such as people, objects, actions, and the overall theme of the picture. Details are provided more fully below.

### SUMMARY OF THE INVENTION

In a first aspect, this invention relates to a method for identifying a subject having abnormal thought patterns such as those commonly but not exclusively found in patients with schizophrenia, the method comprising detecting and measuring thought disorder by:

a) providing the subject with a picture or other stimulus for which salient items have already been identified;

b) causing the subject to verbally describe what he or she observes;

c) transcribing or recording subject's verbal observation; and

d) determining the number and type of differences in vocabulary between the subject's description and a set of words that can denote the previously identified salient items to develop a score of completeness of the subject's description of the picture or stimulus.

In a second aspect, this invention relates to a method for detecting and measuring the severity of thought disorder, the method comprising detecting and measuring thought disorder by:

a) providing the subject with a picture or other stimulus for which salient items have already been identified;

b) causing the subject to verbally describe what he or she observes;

c) transcribing or recording subject's verbal observation; and

d) determining the number and type of differences in vocabulary between the subject's description and a set of words that can denote the previously identified salient items to develop a score of completeness of the subject's description of the picture or stimulus.

### DESCRIPTION OF THE FIGURES

FIG. 1 is an outline of the methodology for detecting abnormalities in verbal description.

FIG. 2 is a graph showing that thought-disordered schizophrenics score differently on the test than healthy controls.

### SPECIFIC EMBODIMENTS

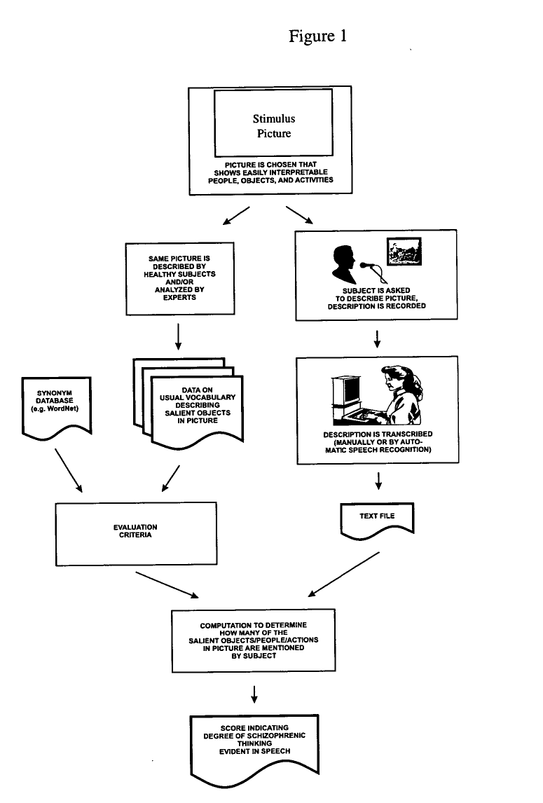

FIG. 1 outlines the process that is used. The subject is given a picture or other stimulus to describe. The subject's verbal responses are recorded and transcribed as a text file.

The vocabulary in the transcribed text is then analyzed by computer to determine how many of the salient items in the picture are mentioned. Salient items means, for example, objects, people, settings, activities, and/or relations that are commonly mentioned by a healthy person describing the picture, as determined by methods described below. On this basis the description is rated for incompleteness, which is indicative of thought disorder.

Technical Details

Picture or Other Stimulus.

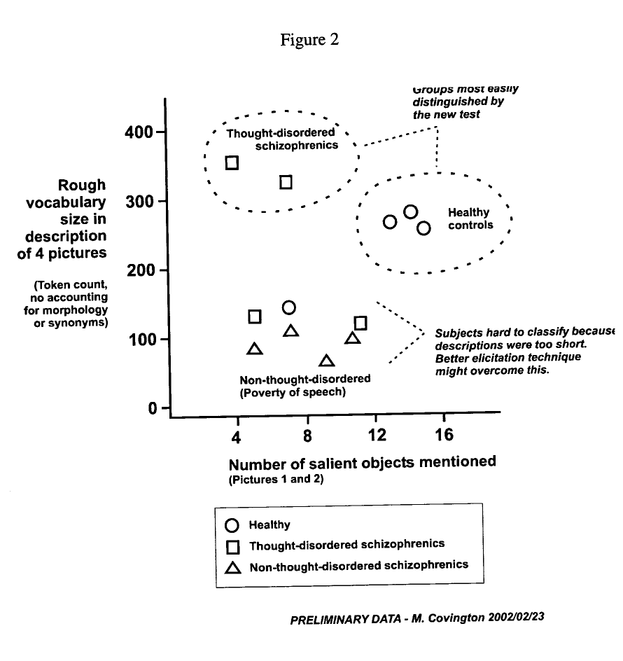

The best pictures for the purpose are those that “tell a story” (like the paintings of Norman Rockwell) and have a clear interpretation. The data in FIG. 2 were collected using pictures in the Thematic Apperception Test as stimuli [Thematic Apperception Test, Harvard University Press: H. Murray; 1971]. However the method of analysis can, in principle, be applied to the verbal responses elicited by any picture that contains easily recognizable representations of objects, plants, animals, people, and/or activities, provided the salient items have previously been identified.

Eliciting the Subject's Description.

The subject is instructed, in a standardized way, to describe the picture or stimulus. The standardized instructions will also specify whether, and to what extent, a laconic subject is to be invited to say more.

Transcription.

The transcription will be done in a standardized way and transcribers will be given standardized instructions. For example, transcribers can be told that the technique requires accurate spelling so that words can be recognized, but does not require accurate punctuation, and that slightly odd pronunciations should be spelled as normal words if they can be recognized.

Identifying Words that Refer to Salient Items.

The crux of the test involves judging how many salient items are mentioned in the subject's description. This is done by counting words that appear to refer to salient items. Accordingly, a suitable set of words is established in advance. Relevant techniques include the following:

Making a list manually, by looking at the picture and perhaps one or more healthy subjects' descriptions of it.

Determining word frequency in a corpus of healthy subjects' descriptions of the picture. The relevant words are content words (nouns, verbs, and adjectives) that occur with considerably higher frequency in the descriptions than in the language as a whole.

Supplementing the word list with a dictionary of synonyms so as to accept all relevant words. The WordNet synonym dictionary [Fellbaum, Christiane, ed. (1998) WordNet: An Electronic Lexical Database, Cambridge, Mass.: MIT Press], freely distributed by Princeton University, is suitable.

Extracting synonyms from a lexical database in advance as part of the preparation of the list of words to be counted.

Using an artificial neural network, genetic algorithm, or other machine learning technique to compare corpora of normal and deviant descriptions, thereby establishing criteria for scoring further descriptions.

Assessing the Result.

The score of a particular subject's description is the number of salient items mentioned, either per se or compared against the length of the description or the size of its vocabulary or repetitiousness, for example. Detailed scoring methods are to be developed, but examples are given below.

The score is determined automatically by computer. Measuring vocabulary size and text size is straightforward, using long-established techniques such as word counting. [Manning, Christopher D., and Schütze, Hinrich, Foundations of Statistical Natural Language Processing (Cambridge, Mass.: MIT Press, 1999), pp. 124-131]. The following is an algorithm for counting salient items mentioned.

### EXAMPLE 1

In this algorithm:

T is a list or array of words in the text, indexed from 1;

S is a list or array of N words denoting salient items, indexed from 1;

F is a list or array of integers with the same number of elements as S;

j, k are local integer variables.

| | |
|---|---|
| 00 | for j := 1 to N do F[j]:= end; |
| 01 | for j := to length(T) do |

| | |
|---|---|
| 02 | for k := 1 to length(S) do |

| | |
|---|---|
| 03 | if T[j] = S[k] then F[k]:= F[k]+1 endif |

| | |
|---|---|
| 04 | end |

| | |
|---|---|
| 05 | end; |
| 06 | score := 0; |
| 07 | for k := to length(S) do |

| | |
|---|---|
| 08 | if F[k] > 0 then score := score + 1 endif |

| | |
|---|---|
| 09 | end; |
| 10 | return score; |

Although written in a pseudocode language resembling Pascal or Modula-3, this example is intended to describe any equivalent implementation in any programming language.

Variations on this algorithm are:

Eliminate prior to line 01 or its equivalent, elements of T that do not appear to be relevant, by some prior criterion such as word length or excessive commonness.

In the word identification step (line 03):

(a) Morphological analysis or stemming is performed on T[j], S[k], or both; and/or

(b) Syntactic parsing is used to disambiguate T[j]; and/or

(c) Any other word sense disambiguation technique is used on T[j]; and/or

(d) T[j] and/or S[k] are mapped onto a set of synonyms such as those identified in WordNet, and synonymy rather than equality is used as the matching criterion; and/or

(e) F[k] is incremented by some value other than 1 depending on how nearly T[j] matches S[k], by a criterion of synonymy or the like; and/or

(f) F[k] is incremented by some value other than 1 depending on whether the relevant word occurred early or late in the text, or before or after prompting by the interviewer.

In line 08 above, score is incremented by some number other than 1 which is a monotone increasing function of F[k].

Use text length, vocabulary size, and/or syntactic complexity as a component of the score.

Use a photograph, a set of pictures, or one or more motion pictures, sculptures, dramatic presentations, or three-dimensional objects as the stimulus, rather than a single drawing.

Use computerized speech recognition for transcription (e.g., ScanSoft, ScanSoft Inc., 9 Centennial Drive, Peabody, Mass. or IBM's ViaVoice).

What is claimed is:

1. A method for identifying a subject having abnormal thought patterns such as those commonly but not exclusively found in patients with schizophrenia, the method comprising detecting and measuring thought disorder by:
a) providing the subject with a picture or other stimulus for which salient items have already been identified;
b) causing the subject to verbally describe what he or she observes;
c) transcribing or recording subject's verbal observation; and
d) determining the number and type of differences in vocabulary between the subject's description and a set of words that can denote the previously identified salient items to develop a score of completeness of the subject's description of the picture or stimulus.

2. A method for detecting and measuring the severity of thought disorder, the method comprising detecting and measuring thought disorder by:
a) providing the subject with a picture or other stimulus for which salient items have already been identified;
b) causing the subject to verbally describe what he or she observes;
c) transcribing or recording subject's verbal observation; and
d) determining the number and type of differences in vocabulary between the subject's description and a set of words that can denote the previously identified salient items to develop a score of completeness of the subject's description of the picture or stimulus.

3. The method of claim 1 wherein the text length, vocabulary size, and/or syntactic complexity in the subject's observations are used as components of the score.

4. The method of claim 1 wherein the stimulus uses an image of any type (drawing, photograph, digital image, projected image, hologram, etc.), set of still pictures, one or more motion pictures, sculptures, dramatic presentations, or three-dimensional objects.

5. The method of claim 1 wherein the subject's observations are captured using computerized speech recognition rather than manual transcription.

6. The method of claim 2 wherein the text length, vocabulary size, and/or syntactic complexity in the subject's observations are used as components of the score.

7. The method of claim 2 wherein the stimulus uses an image of any type (drawing, photograph, digital image, projected image, hologram, etc.), set of still pictures, one or more motion pictures, sculptures, dramatic presentations, or three-dimensional objects.

8. The method of claim 2 wherein the subject's observations are captured using computerized speech recognition rather than manual transcription.
